(19) United States
(12) Patent Application Publication
(10) Pub. No.: US 2007/0249966 A1
(43) Pub. Date: Oct. 25, 2007

(54) Device and method for measuring urethral angle

(76) Inventor: Mark Ira Hunter, Orange, CA (US)

Correspondence Address: JAFARI LAW GROUP, P.C., 801 N. PARKCENTER DRIVE, SUITE 220, SANTA ANA, CA, 92705, US

(21) Appl. No.: 11/406,628

(22) Filed: Apr. 19, 2006

(51) Int. Cl. A61B 5/103

(52) U.S. Cl. 600/587

(57) ABSTRACT

A medical device for measuring the angular deviation of an elongated insertion instrument inserted into an urethra and method for determining the condition of the pelvic floor of a patient. It includes a channel adapted to receive an elongated insertion instrument along a longitudinal access and an indicator to measure an angular deviation along a longitudinal axis from a reference axis. The medical device may also include an elongated insertion instrument inserted along the longitudinal access of its channel. A method for determining the condition of the pelvic floor of a patient by inserting the medical device into the urethra of a patient, reading a first angular deviation from a reference axis, instructing the patient to strain or cough, and reading a second angular deviation from a reference axis. The difference in first and second angular deviation measures the degree of prolapse of the urethrovesical junction of the patient.

### BACKGROUND

1. Field of the Invention

The present invention relates in general to medical devices and more specifically to medical devices for determining degree of prolapse of the urethrovesical junction of a patient.

2. Background of the Invention

Physicians use a procedure commonly referred to as the “Q-tip® Test” for determining the condition of the female pelvic floor and the degree of prolapse of the urethrovesical junction. The procedure utilizes a soft or cotton-tipped instrument similar to the device known as a Q-tip®. This instrument differs however from the standard cosmetic cotton swab in that it has a long wooden shaft and a cotton swab at only one end. The test is routinely performed on patients who have reported urinary incontinence or prolapse of the pelvic organs into the vagina.

To perform the test, the patient is placed flat on her back with her feet in stirrups. The instrument is then inserted into the urethra and advanced to the junction between the urethra and the bladder. Using a standard protractor, the resting angle is measured as the angle between the instrument and the plane of the floor. The patient is then asked to strain or cough, thereby increasing the intra-abdominal pressure and, in certain patients, causing the angle between the urethral axis, or the instrument, and the floor to change. The new angle is then recorded and a diagnosis is made on the basis of the degree of angular deviation.

Although the test has been used for several decades, the basic procedure and equipment have remained the same. Both the resting and the straining angles are measured either by visual approximation, or by use of a standard protractor. In the first example, visual estimation of the angle between the instrument and the floor can be problematic in that the instrument can be as far as three or four feet from the floor, thus making establishment of the horizontal reference difficult. In addition, the visual estimation and resolution of an angular difference is largely dependent on the skill of the examiner in interpreting spatial orientation.

To solve the above-mentioned problems, the incorporation of a standard protractor has become popular. In this version of the Q-tip® test, the protractor is held up next to the deviating cotton-tipped instrument at rest and with strain. The numerical difference in angle between the instrument and the floor can be approximated by gazing at the protractor in close approximation with the instrument. This method also introduces error in measurement, first by incorporating a numerical scale that is only in close approximation to the instrument. Error is then introduced in the estimation of the instrument projection onto a neighboring protractor.

In addition, the protractor is placed against the instrument with the zero reference arm of the protractor assumed to be parallel to the floor by gross approximation. Again, as the floor is often as far as four feet from the instrument, error can be introduced in the assumption that the bottom of the protractor is in fact parallel to the floor. The degree of this error is inversely proportional to the operator's skill in spatial orientation and manipulation. Further, depending on the type and size of the protractor used, the entire process can be awkward and uncomfortable. Lastly, the use of a non-disposable protractor requires that the examiner remove contaminated examination gloves before touching the protractor, and replacing the gloves once the test has been completed.

### SUMMARY OF THE INVENTION

To overcome the limitations in the prior art, and to overcome other limitations that will become apparent upon reading and understanding the present specification, the present invention provides a medical device for measuring the angular deviation of an elongated insertion instrument inserted into an urethra and method for determining the condition of a pelvic floor of a patient and whether prolapse of a urethrovesical junction exists in a patient.

A medical device in accordance with the present invention includes an indicator of angular deviation that contains a channel for accepting an elongated insertion instrument along a longitudinal axis. The indicator is used to determine the angular deviation of a longitudinal axis of the insertion instrument from a reference axis.

In a further aspect of the present invention, the medical device may include an elongated insertion instrument for insertion into the urethra, wherein the insertion instrument is generally inflexible, an indicator attached to the elongated insertion instrument, comprising indicator marks for determining an angle of the insertion instrument with an indicator, where the indicator maintains its position with respect to the insertion instrument as the insertion instrument tilts from a horizontal position, and a pointer, attached to the indicator, where the pointer continues to point towards a reference axis as the insertion instrument and pointer tilt from the reference axis position.

It is an object of the present invention to provide sterile, single-use devices that are easily disposable. It is another object of the present invention to reduce the error in approximating the angle of the insertion instrument against the angle of the floor, or horizontal plane. It is still another object of the invention to provide a small, light, and easy to use device that is comfortable to use.

A method in accordance with the present invention comprises laying the patient flat on her back, inserting the medical device into her urethra, advancing the urethral angle medical device into a junction between the urethra and the bladder, or the vesicourethral junction of the patient, and recording the angle shown by the medical device. Then, instructing the patient to cough or strain and record the second angle shown by the medical device. The difference between the first and second reading is a measure of the degree of prolapse of the vesicourethal junction.

### BRIEF DESCRIPTION OF THE DRAWINGS

Referring now to the drawings in which like reference numbers represent corresponding parts throughout:

FIG. 1 illustrates a side planar view of an embodiment of the medical device;

FIG. 2 illustrates a side planar view of an embodiment of the medical device when the medical device is tilted;

FIG. 3 illustrates an enlarged planar view of the indictor region shown in FIG. 2;

FIG. 4 illustrates a printed indicator template;

FIG. 5 illustrates a side planar view of the printed indicator template of FIG. 4, folded in half with a pivot region;

FIG. 6 illustrates a front planar view of the printed indicator template of FIG. 5;

FIG. 7 illustrates a front planar view of a pointer;

FIG. 8 illustrates a front planar view of an embodiment of the medical device;

FIG. 9 illustrates an enlarged side planar view shown in FIG. 1;

FIG. 10 illustrates a top planar view of an embodiment of the medical device;

FIG. 11 illustrates a bottom planar view of an embodiment of the medical device;

FIG. 12 illustrates a side perspective side view of an embodiment of the medical device;

FIG. 13 illustrates another side perspective view of an embodiment of the medical device; and

FIG. 14 illustrates a side planar view of an embodiment of the medical device when the medical device is tilted.

### DETAILED DESCRIPTION OF THE PRESENTLY PREFERRED EMBODIMENTS

In the following description of the preferred embodiment, reference is made to the accompanying drawings that form a part hereof, and in which is shown by way of illustration a specific embodiment in which the invention may be practiced. It is to be understood that other embodiments may be utilized and structural changes may be made without departing from the scope of the present invention.

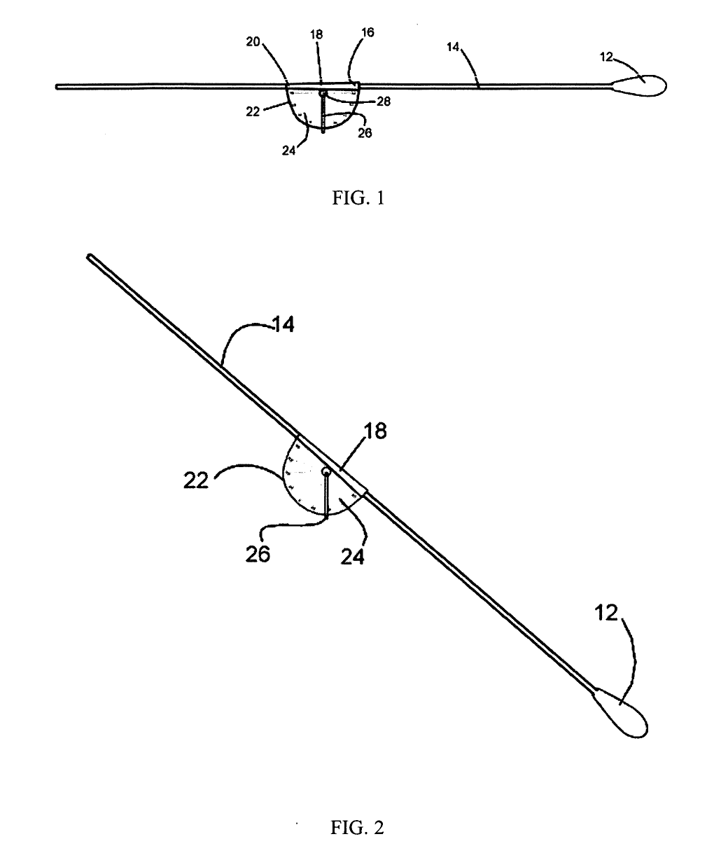

FIG. 1 illustrates a side planar view of an embodiment of the medical device, with elongated insertion instrument 14, elongated insertion instrument tip 12, channel opening 16, channel 18, channel opening 20, indicator 22, angle indicators 24, pointer 26, and pivot region 28.

A preferred method of determining the degree of prolapse of a patient's urethrovesical junction involves inserting elongated insertion instrument 14 into the urethra, to the opening of the bladder, of a patient. The insertion instrument represents a continuation of the urethral axis. With the patient at rest, a first angle is read from indicator 22. The patient is then asked to strain, as in a Valsalva maneuver, and while the patient continues to strain a second angle is read from indicator 22. The angular deviation between the first and second angle readings is a measure of the degree of prolapse of the patient's urethrovesical junction.

The medical device and method for measurement of the urethral angle employ the use of gravity in the establishment of a horizontal reference point. That reference angle would then be displayed on indicator 22, thereby removing the error of simply gazing at a Q-tip®, or other instrument, using the standard techniques. An embodiment is shown in FIG. 1, with elongated insertion instrument 14 parallel with the floor.

When the insertion instrument 14 is inserted into the patient's urethra, at rest, the shaft will likely be somewhere close to parallel with the floor. In this position, as in FIG. 1, the pointer 26 will be pointing towards the floor, as it is suspended from pivot region 28 in a gravity-dependent fashion.

FIG. 2 illustrates a side planar view of an embodiment of the medical device according to the present invention when the medical device is tilted, with insertion instrument 14, insertion instrument tip 12, channel 18, indicator 22, angle indicators 24, and pointer 26.

When the patient bears down, or performs a Valsalva maneuver, the angle of insertion instrument 14 will tilt upwards. As insertion instrument 14 raises its position, indicator 22 will maintain its position with respect to insertion instrument 14. Pointer 26, however, will not maintain a fixed angle with respect to insertion instrument 14, but instead will continue to point straight down towards the floor since pointer 26 is suspended from pivot region 28.

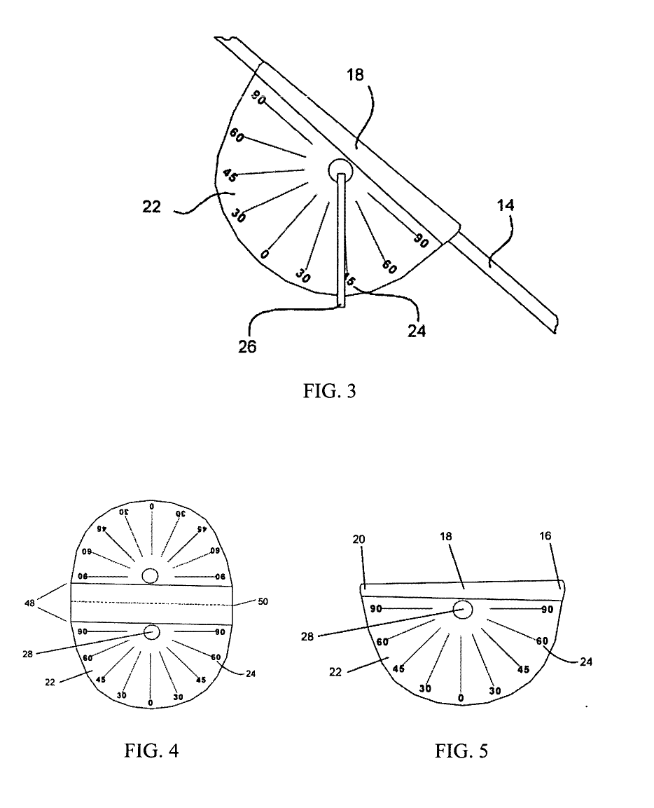

The same embodiment of the medical device is shown in FIG. 3 as an enlarged planar view of the indicator region, with insertion instrument 14, channel 18, indicator 22, angle indicators 24, and pointer 26.

The angle deviation from the reference axis is then read by reading one of the angles that pointer 26 points to on indicator 22. The position of the pointer 26 will indicate the angle at which insertion instrument 14 is tilted with respect to a reference axis, and the angle indicators 24 determine the appropriate angle. In the embodiment shown, the user would estimate the angle within the nearest 5 degrees. Alternatively, a larger indicator 22 with more angle indicators 24 would increase the accuracy of the measurement. The size of indicator 22 and the number of angle indicators 24 can vary in many different ways without departing from the scope of the present invention.

In the embodiment shown in FIG. 3, when insertion instrument 14 is parallel to the floor, or completely horizontal, pointer 26 should point towards zero degrees on the angle indicators 24, as it is in FIG. 1. The actual numbers of angle indicators 24 can vary without departing from the scope of the present invention. For example, the zero degree angle can be labeled as 360 degrees, or 180 degrees. Moreover, angle indicators 24 can be labeled with words such as “level,” or “prolapse,” or not be labeled at all. To put it simply, angle indicators 24 should be labeled well enough so that a user of the medical device should be able to look at the position of pointer 26 and determine the angle that insertion instrument 14 is tilted at.

A printed template of indicator 22 is illustrated in FIG. 4, with indicator 22, angle indicators 24, pivot region 28, channel base fold line 48, and channel top fold line 50.

Indicator 22 can be folded along channel top fold line 50, creating a protractor-type surface on both sides of indicator 22. Indicator 22 is preferably made of paper, wood, plastic, or other lightweight, sterile, cost effective, disposable material. It is important that indicator 22 be lightweight, so as not to bend insertion instrument 14 when attached to insertion instrument 14. Alternatively, insertion instrument 14 can be made from a firmer material so that it does not bend when indicator 22 is attached.

In a preferred embodiment, indicator 22 is folded around insertion instrument 14, and then the two channel base fold lines 48 are then fixed together. The two sides of indicator 22 can be fixed together using glue, staples, or some other adhesive. In addition to attaching both sides of indicator 22 to one another, the adhesive can attach indicator 22 directly to insertion instrument 14 without departing from the scope of the present invention.

Following fixation of the two halves of indicator 22, the manufacturer can punch out pivot region 28, where pointer 26 is inserted. In a preferred embodiment of the invention, pivot region 28 is punched at a central point under bottom fold lines 48. Pivot region 28 can be punched prior to folding indicator 22, or can be created in any other manner without departing from the scope of the present invention.

FIG. 5 illustrates a side planar view of the printed template of FIG. 4, folded in half, with channel 18, channel opening 20, channel opening 16, indicator 22, angle indicators 24, and pivot region 28.

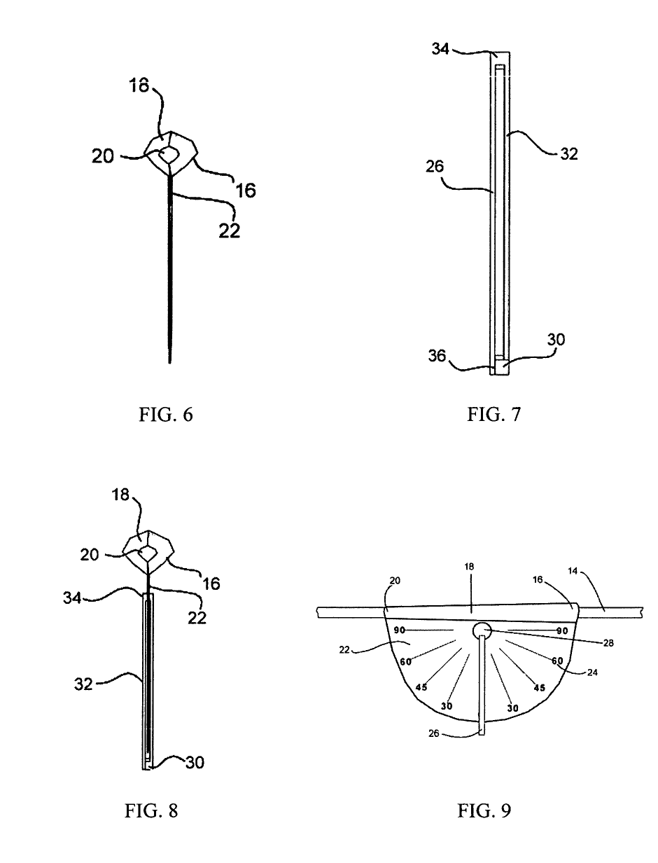

The front planar view of the printed template of FIG. 5 is shown in FIG. 6, with channel 18, channel opening 20, channel opening 16, and indicator 22. FIG. 6 shows in greater detail exactly how indicator 22 is folded to create channel opening 20 and channel opening 16.

A front planar view of pointer 26, is shown in FIG. 7, with pointer top 34, pointer side 32, pointer junction 36, and pointer bottom 30.

In an embodiment of the present invention, pointer bottom 30 is heavier than pointer top 34, so that pointer 26 will tend to swing when insertion instrument 14 is tipped from the reference axis. In an embodiment of the present invention, pointer 26 separates as shown in the diagram at pointer junction 36, so as to insert into pivot region 28. Pointer junction 36 can be located at another location other than near the pointer bottom 30 without departing from the scope of the present invention.

The important function of pointer 26 is that it is freely suspended from pivot region 28 so as to always point downwards with gravity as insertion instrument 14 is being tipped. Pointer 26 can be a loop, ring, or other gravity influenced device without departing from the scope of the present invention. For example, pointer 26 can be as simple as a ball hanging from a string. Or indicator 22 can be turned upside-down, and pointer 26 can be a metronome-like device with a weight on the bottom and a pointer at the top. There are many ways to create pointer 26 without departing from the scope of the present invention.

A front planar view of pointer 26, as shown in FIG. 4, attached to indicator 22, as show in FIG. 6, is shown in FIG. 8, with channel 18, channel opening 16, channel opening 20, indicator 22, pointer top 34, pointer side 32, and pointer bottom 30. FIG. 8 illustrates one embodiment of how pointer 26 can be attached to indicator 22.

A planar view of the medical device of FIG. 3 is shown in FIG. 9, with insertion instrument 14, channel opening 16, channel 18, channel opening 20, indicator 22, angle indicators 24, pointer 26, and pivot region 28.

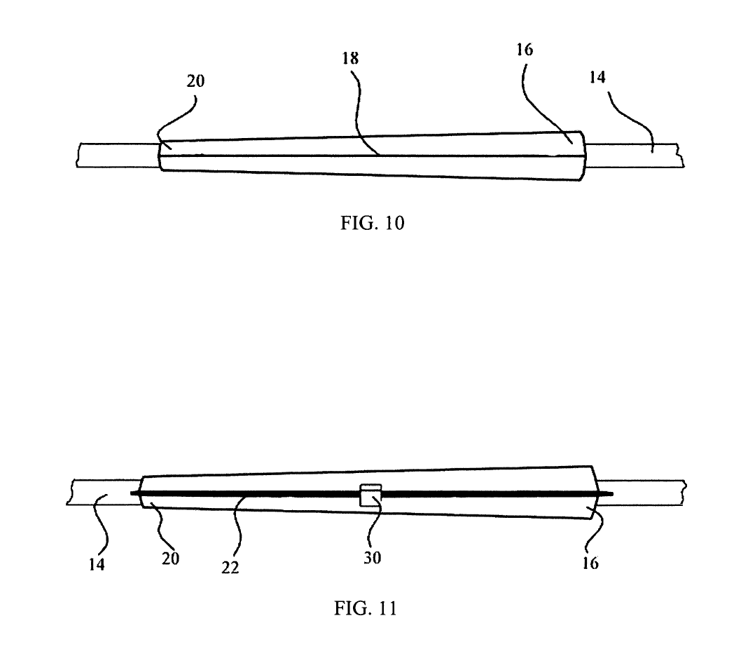

A top planar view of an embodiment of the medical device of FIG. 3 is shown in FIG. 10, with insertion instrument 14, channel 18, channel opening 16, and channel opening 20.

A bottom planar view of the medical device of FIG. 3 is shown in FIG. 11, with insertion instrument 14, channel opening 16, channel opening 20, indicator 22, and pointer bottom 30.

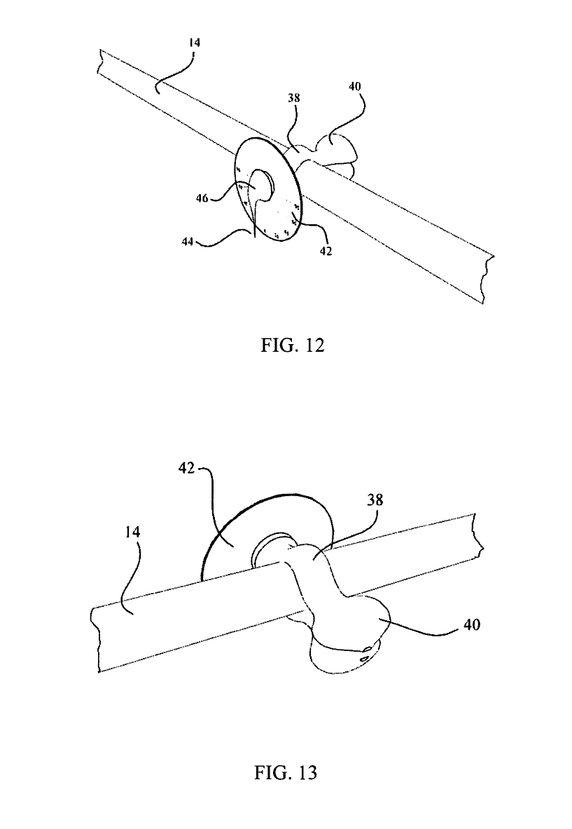

Other embodiments can also be employed to arrive at the same utility. Another embodiment may have an angle pointer firmly attached to insertion instrument 14, while the indicator freely hangs towards the ground. An example of this embodiment is shown in FIG. 12, with insertion instrument 14, bracket 38, bracket counterbalance 40, bracket shaft 46, pointer 44, and indicator 42.

In this embodiment, pointer 44 is firmly attached to bracket 38, which in turn is firmly attached to insertion instrument 14. When insertion instrument 14 tilts from the horizontal position, pointer 44 tilts with insertion instrument 14. Indicator 42 is free to rotate along the axis made by bracket shaft 46. Indicator 42 is weighted towards the floor, and is free to rotate about bracket shaft 46, maintaining a constant position with respect to the gravitational pull. The angle of deviation of insertion instrument 14 with respect to the reference axis is then read by reading the number on indicator 42 that pointer 44 points to.

An opposite side perspective view of the medical device of FIG. 12 is shown in FIG. 13, with insertion instrument 14, bracket 38, bracket counterbalance 40, and indicator 42.

In situations where the gravity-dependent device is only located on one side of insertion instrument 14, like the current embodiment, a counter-balance system may have to be used to keep the medical device balanced. Otherwise, the insertion instrument might turn while in the patient's urethra. In the current embodiment, bracket counterbalance 40 is used to offset the additional weight on the other side of insertion instrument 14 caused by indicator 42. Bracket 38 and bracket counterbalance 40 are used in this embodiment so as to be able to easily move the medical device up and down the shaft of insertion instrument 14.

Other ways of fastening bracket 38 to insertion instrument 14 can be used without departing from the scope of the present invention. For example, bracket 38 could simply be glued to insertion instrument 14 if indicator 42 is light enough.

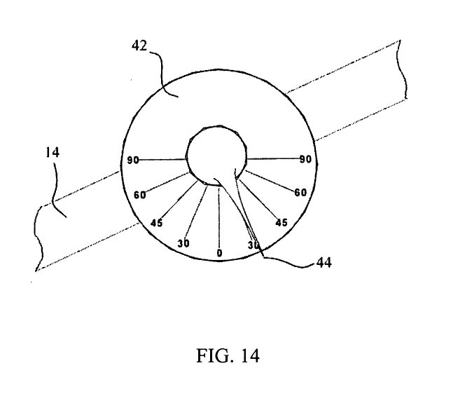

FIG. 14 illustrates a side planar view of an embodiment of the medical device shown in FIG. 12 when the medical device is tilted, with insertion instrument 14, pointer 44, and indicator 42. As shown in FIG. 14, when insertion instrument 14 is tilted, pointer 44 tilts with it. Indicator 42 is weighted at the zero angle, so that it rotates when insertion instrument 14 tilts in one direction or the other. The angle of deviation of insertion instrument 14 with respect to the horizontal plane is then read by reading the number on indicator 42 that pointer 44 points to.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims.

What is claimed is:

1. A medical device, comprising:
(a) a channel adapted to receive an elongated insertion instrument along a longitudinal axis of said channel; and
(b) an indicator adapted to indicate an angular deviation of said longitudinal axis of said channel from a reference axis.

2. The medical device of claim 1, wherein said reference axis is oriented substantially horizontal.

3. The medical device of claim 1, wherein said indicator comprises:
(a) a scale to indicate a range of angular deviations; and
(b) a pointer suspended about a pivot region, and adapted to point to said indicated angular deviation of said longitudinal axis of said channel from said reference axis.

4. The medical device of claim 3, wherein said pointer indicates an angular deviation of approximately zero when said longitudinal axis of said channel is substantially the same as said reference axis.

5. The medical device of claim 3, wherein said range of angular deviation extends from −90 to +90 degrees.

6. The medical device of claim 3, wherein said range of angular deviation extends from approximately 180 degrees.

7. The medical device of claim 1, wherein said indicator comprises:
(a) a scale to indicate a range of angular deviations, wherein said scale is suspended about a pivot region; and
(b) a pointer adapted to point to said indicated angular deviation of said longitudinal axis of said channel from said reference axis.

8. The medical device of claim 7, wherein said pointer points to an angular deviation of approximately zero when said longitudinal axis of said channel is substantially the same as said reference axis.

9. The medical device of claim 7, wherein said indicator further comprises a counterbalance located on the opposite side of said indicator on said elongated insertion instrument.

10. The medical device of claim 7, wherein said range of angular deviation extends from −90 to +90 degrees.

11. The medical device of claim 7, wherein said range of angular deviation extends from approximately 180 degrees.

12. A medical device, comprising:
(a) an elongated insertion instrument;
(b) a channel through which said elongated insertion instrument is inserted along a longitudinal axis of said channel; and
(c) an indicator adapted to indicate an angular deviation of said longitudinal axis of said channel from a reference axis.

13. The medical device of claim 12, wherein said elongated insertion instrument is adapted to be inserted into a urethra of a subject.

14. The medical device of claim 12, wherein said elongated insertion instrument comprises a cotton swab situated at a distal end thereof.

15. The medical device of claim 14, wherein said indicator is situated at a proximal end of said elongated insertion instrument.

16. The medical device of claim 12, wherein said reference axis is oriented substantially horizontal.

17. The medical device of claim 12, wherein said indicator comprises:
(a) a scale to indicate a range of angular deviations; and
(b) a pointer suspended about a pivot region, and adapted to point to said indicated angular deviation of said longitudinal axis of said channel from said reference axis.

18. The medical device of claim 17, wherein said pointer indicates an angular deviation of approximately zero when said longitudinal axis of said channel is substantially the same as said reference axis.

19. The medical device of claim 17, wherein said range of angular deviation extends from −90 to +90 degrees.

20. The medical device of claim 17, wherein said range of angular deviation extends from approximately 180 degrees.

21. The medical device of claim 12, wherein said indicator comprises:
(a) a scale to indicate a range of angular deviations, wherein said scale is suspended about a pivot region; and
(b) a pointer adapted to point to said indicated angular deviation of said longitudinal axis of said channel from said reference axis.

22. The medical device of claim 21, wherein said pointer points to an angular deviation of approximately zero when said longitudinal axis of said channel is substantially the same as said reference axis.

23. The medical device of claim 21, wherein said indicator further comprises a counterbalance located on the opposite side of said indicator on said elongated insertion instrument.

24. The medical device of claim 21, wherein said range of angular deviation extends from −90 to +90 degrees.

25. The medical device of claim 21, wherein said range of angular deviation extends from approximately 180 degrees.

26. A method of measuring a degree of prolapse of an urethrovesical junction of a subject, comprising:
(a) inserting an elongated insertion instrument into a urethra of said subject;
(b) reading an indicator on said elongated insertion instrument while said subject is at rest, wherein said indicator indicates a first angular deviation of a longitudinal axis of said elongated insertion instrument from a reference axis;
(c) instructing said subject to perform a Valsalva maneuver; and
(d) reading said indicator on said elongated insertion instrument while said subject is straining or coughing, wherein said indicator indicates a second angular deviation of said longitudinal axis of said elongated insertion instrument from said reference axis;
wherein a difference between said first angular deviation and said second angular deviation is a measure of said degree of prolapse of said urethrovesical junction of a subject.

27. The method of claim 26, wherein said inserting said elongated insertion instrument comprises inserting said elongated insertion instrument to approximately a junction between said urethra and a bladder.

28. The method of claim 26, wherein said elongated insertion instrument comprises a cotton swab situated at a distal end thereof.

29. The method of claim 28, wherein said indicator is situated at a proximal end of said elongated insertion instrument.

30. The method of claim 26, wherein said indicator comprises:
(a) a scale to indicate a range of angular deviations; and
(b) a pointer suspended about a pivot region, and adapted to point to said indicated angular deviation of said longitudinal axis of said channel from said reference axis.

31. The method of claim 26, wherein said indicator comprises:
(a) a scale to indicate a range of angular deviations, wherein said scale is suspended about a pivot region; and
(b) a pointer adapted to point to said indicated angular deviation of said longitudinal axis of said channel from said reference axis.

32. The method of claim 31, wherein said indicator further comprises a counterbalance located on the opposite side of said indicator on said elongated insertion instrument.
